(19) United States
(12) Patent Application Publication
(10) Pub. No.: US 2011/0256266 A1
(43) Pub. Date: Oct. 20, 2011

(54) COMPOSITION FOR MAKING A DAIRY PRODUCT

(76) Inventors: Brian J. Orme, Logan/River Heights, UT (US); Randall Kirk Thunell, Logan, UT (US); Paul Gerard Bruinenberg, Udenhout (NL)

(21) Appl. No.: 13/124,914

(22) PCT Filed: Nov. 2, 2009
(86) PCT No.: PCT/EP2009/064429

§ 371 Date: Jul. 1, 2011

Related U.S. Application Data
(60) Provisional application No. 61/110,145, filed on Oct. 31, 2008.

(30) Foreign Application Priority Data
Mar. 2, 2009 (EP) 09154134.2

(51) Int. Cl. A23C 9/12; C12N 1/20

(52) U.S. Cl. 426/42; 435/252.9

(57) ABSTRACT

The present invention relates to improved bulk starter cultures and their use in the manufacture of fermented products. The present invention also relates to methods for making the improved bulk starter cultures.

### FIELD OF THE INVENTION

The present invention is in the field of production of fermented products. More in particular, it relates to improved compositions for making dairy products, in particular improved bulk starter compositions.

### BACKGROUND OF THE INVENTION

Microorganisms are involved in the production of many food and feed products including fermented dairy products such as cheese, yoghurts, sour cream, kefir, butter and koumiss. Selected strains of microorganisms initiating and carrying out the desired fermentation essential in the manufacture of the above products are often referred to as starter cultures. In particular, cultures of lactic acid bacteria are widely used as starter cultures.

Lactic acid bacteria commonly used in the food industry can be divided into mesophilic lactic acid bacteria including the genera Lactococcus, Leuconostoc and Pediococcus and thermophilic lactic acid bacteria including the genera Streptococcus and Lactobacillus. While mesophilic lactic acid bacteria have optimum growth temperatures of about 30° C., thermophilic lactic acid bacteria have optimum growth temperatures of about 40° C. to about 45° C.

Starter cultures are generally available from commercial manufacturers in lyophilized, frozen or liquid form. They can comprise only a single lactic acid bacterium species, but can also be mixed cultures comprising two or more different lactic acid bacterium species. Mixed starter cultures are often used to minimise bacteriophage infection.

Starter cultures can be inoculated directly into milk without intermediate transfer and/or propagation. Such starter cultures are generally referred to as direct vat set (DVS) or direct to vat inoculation (DVI) cultures. Despite the availability of DVS and DVI cultures, it is not uncommon that dairies produce in-house bulk starter cultures. Bulk starter cultures are made by inoculating a growth medium using a small amount of a starter culture followed by incubating the growth medium under conditions permitting the bacteria to propagate for a sufficient period of time to provide a desired cell number. The obtained bulk starter culture is then used to inoculate milk for the manufacture of fermented dairy products.

Acidity is one of the major control factors in the making of cheese and other dairy products and is developed by adding a starter culture of lactic acid bacteria to milk. Acid production is the major function of the starter bacteria. The bacteria ferment lactose present in the milk to produce lactic acid. Failure of the starter culture to develop acidity at a sufficient rate can result in allowing harmful or undesirable organisms to grow. It can also result in taint, high moisture and a weak curd in the cheese. Moreover, acid development contributes to proteolysis and flavor production in cheese and affects rheological properties of cheese. Too much acidity developing too quickly can result in cheese with poor body and texture. The correct development of controlled acidity is therefore critical to the good manufacture of cheese and to inhibit the proliferation of harmful and spoilage organisms.

It has been observed that cells of starter cultures have a reduced viability and consequently rapidly loose their metabolic activity, such as e.g. their acidification activity, when they are subjected to stress during harvesting, packaging and storage even for short periods of time.

U.S. Pat. No. 6,660,515 describes that the acidifying activity of starter cultures can be improved by cultivating the starter culture in association with a lactic acid bacterial helper organism which is defective in its pyruvate metabolism. A disadvantage of this method is that it is laborious and time-consuming, since helper organisms first have to be prepared by means of mutagenesis and thereafter selected and tested for the desired property.

In view of the importance of acidity in the preparation of cultured dairy products, such as e.g. cheese, there is a continuing need in the art to provide starter cultures having improved acidification activities. The present invention addresses this need.

### SUMMARY OF THE INVENTION

It was found in accordance with the present invention that a mixed bulk starter culture having an increased acidification activity can be prepared by introducing at least two different concentrated Lactococcus lactis strains into a growth medium, incubating the obtained growth medium to prepare a mixed bulk starter culture and selecting a mixed bulk starter culture having an acidification activity of at least 1.45 ΔpH/2.5 h at 31° C. The invention also provides methods for making a fermented product by adding the improved bulk starter cultures to a substrate such as milk and fermenting the substrate to produce the fermented product.

### DETAILED DESCRIPTION OF THE INVENTION

In a first aspect the invention relates to a method for preparing a mixed bulk starter culture, in particular a method for preparing a high activity mixed bulk starter by using concentrated starter cultures, the method comprising the steps of introducing at least two different concentrated Lactococcus lactis strains into a growth medium, incubating the obtained growth medium to prepare a mixed bulk starter culture and selecting a mixed bulk starter culture having an acidification activity of at least 1.45 ΔpH/2.5 h at 31° C. “Starter culture” as used herein refers to a preparation containing microbial cells that is intended for inoculating a medium to be fermented. “Bulk starter culture” as used herein means a starter culture propagated at a dairy plant for inoculation into a substrate, e.g. milk. Incubation is done at a temperature conducive to the growth of the microbial starter organisms for a period of time until the desired cell concentration and activity of the bulk starter culture are reached. Incubation can be stopped by cooling to e.g. below 10° C. In the introduction step the growth medium is inoculated with the concentrated Lactococcus lactis strains and in the incubation step the Lactococcus lactis strains are grown/propagated in the growth medium. In other words, in the introduction step an inoculated medium is produced and in the incubation step the inoculated medium is ripened to produce a mixed bulk starter culture. In the selection step a mixed bulk starter culture having an acidification activity of at least 1.45 ΔpH/2.5 h at 31° C. is selected for further use, e.g. in a method for making a fermented product such as a method for making a fermented product as described herein. A mixed bulk starter cultures having an acidification activity of at least 1.45 ΔpH/2.5 h at 31° C. has sufficient acidification activity to be used in a method for making a fermented product such as a method for making a fermented product as described herein. A mixed bulk starter culture having an acidification activity of less than 1.45 ΔpH/2.5 h at 31° C. is not selected, as its acidification activity is insufficient for it to be used in a method for making a fermented product such as a method for making a fermented product as described herein. In cheese factories the cheese making process is strictly regulated with respect to the timing of various process steps. A high activity starter culture with constant, and thus predictable, performance plays a crucial role in this. An activity of at least 1.45 ΔpH/2.5 h at 31° C. is needed to ensure an efficient process for the preparation of high quality cheese. A starter having a low acidification activity, i.e. an activity of less than 1.45 ΔpH/2.5 h at 31° C., causes delay in the cheese making process steps and affects the final pH of the cheese, and in relation to that the moisture content of the cheese as well. It is well known in the art that a varying moisture content in cheese affects the sensory quality of the cheese. Selection can be done based on acidification activity measurements. Acidification activity can be measured as described herein. If desired, selection can be done by measuring the acidification activity of a starter culture, followed by testing the actual performance of the starter culture with the desired acidification activity (i.e. an acidification activity of at least 1.45 ΔpH/2.5 h at 31° C.) in a cheese test vat (i.e. the activity results provide a starting place for an inoculum level of the starter culture in the cheese making process; the level is refined based on actual cheese making performance of the starter culture).

In an embodiment the growth medium is incubated for less than ten hours, preferably less than nine hours, more preferably less than eighth hours, and in particular less than seven hours. Preferably, the incubation period is between 1 and 10 hours, more preferably between 2 and 9 hours, even more preferably between 3 and 8 hours and most preferably between 4 and 7 hours.

Optionally, after the incubation and/or selection step cells in the obtained mixed bulk starter culture can be recovered/harvested/isolated/separated from the growth medium. Methods to recover/harvest/isolate/separate microbial cells from growth media are well known to the man skilled in the art and include centrifugation, (ultra)filtration or combinations thereof to name just a few. After recovering, harvesting, isolating and/or separating the cells by centrifugation and/or (ultra)filtration, they can be packaged for subsequent use or storage. The cells in the obtained mixed bulk starter cultures can also be aliquoted for subsequent use or storage without previously being recovered/harvested/isolated/separated.

Suitable strains are strains of Lactococcus lactis subspecies including, but not limited to, Lactococcus lactis subsp. lactis, Lactococcus lactis subsp. lactis biovar. diacetylactis, Lactococcus lactis subsp. hordniae and Lactococcus lactis subsp. cremoris. In an embodiment one of the at least two different Lactococcus lactis strains is a Lactococcus lactis subsp. cremoris strain. The other Lactococcus lactis strain can be any Lactococcus lactis strain with the exception of the specific Lactococcus lactis subsp. cremoris strain. In other words, when two Lactococcus lactis subsp. cremoris strains are introduced into the growth medium, the strains must differ.

An advantage of using mixed bulk starter cultures comprising at least one Lactococcus lactis subsp. cremoris strain is that during the cheese, e.g. Cheddar cheese, manufacturing process the cell number of a Lactococcus lactis subsp. cremoris strain can be better controlled than the cell number of a Lactococcus lactis subsp. lactis strain. Lactococcus lactis subsp. lactis strains are less sensitive towards high temperature and high salt in comparison with Lactococcus lactis subsp. cremoris, and therefore Lactococcus lactis subsp. lactis grow to higher cells numbers in cheese. High cell numbers of Lactococcus lactis subsp. lactis in cheese, e.g. Cheddar cheese, can result in off-flavour production, e.g. development of bitterness, during cheese maturation.

Besides the at least two different Lactococcus lactis strains, the mixed bulk starter culture may comprise further microbial strains such as e.g. lactic acid bacteria strains. Mixed bulk starter cultures as such are prepared by introducing the further microbial strains, such as e.g. lactic acid bacteria strains, into the growth medium and preparing the mixed bulk starter cultures. Preferably, the further microbial strains are introduced into the growth medium in concentrated form. “Lactic acid bacteria” as used herein refers to Gram-positive, low-GC, acid tolerant, generally non-sporulating, non-respiring rod or cocci that are associated by their common metabolic and physiological characteristics. They ferment carbohydrates to produce acids, including lactic acid as the predominantly produced metabolic endproduct. They constitute a heterogeneous group including, but not limited to, the genera Aerococcus, Carnobacterium, Enterococcus, Lactobacillus, Lactococcus, Leuconostoc, Oenococcus, Pediococcus, Sporolactobacillus, Streptococcus, Tetragenococcus, Vagococcus, and Weisella. Besides lactic acid bacteria strains, the mixed bulk starter culture may also comprise other bacterial strains including, but not limited to, strains of the genera Bifidobacterium, Corynebacteria such as Brevibacterium, Micrococcus and Propionibacterium. Besides the above bacterial strains other microbial strains such as strains of molds, fungi and/or yeasts including, but not limited to, the genera Penicillium, Geotrichum, Saccharomyces and Kluyveromyces may be included in the mixed bulk starter cultures.

A bulk starter culture according to the invention can be referred to as a mixed bulk starter culture, as it comprises two or more different Lactococcus lactis strains (e.g. two Lactococcus lactis strains and one Streptococcus salivarius thermophilus strain). It can be referred to as a mixed mesophilic bulk starter culture, when it consists of only mesophilic bacteria (e.g. two Lactococcus lactis strains and one Leuconostoc mesenteroides cremoris strain).

In an embodiment the different strains in the bulk starter culture, such as e.g. at least two different Lactococcus lactis strains, are introduced separately into the growth medium. Alternatively, all or some of the strains can first be co-formulated and thereafter the co-formulation can be introduced into the growth medium. For instance, a mixed bulk starter culture comprising a Lactococcus lactis subsp. lactis strain, a Lactococcus lactis subsp. cremoris strain and a Streptococcus salivarius thermophilus strain can be made by introducing the three different strains separately into the growth medium, but can also be made by introducing a formulation comprising all three strains into the growth medium. It can even be made by introducing a formulation comprising the Lactococcus lactis subsp. lactis strain and the Lactococcus lactis subsp. cremoris strain and a formulation comprising the Streptococcus salivarius thermophilus strain into the growth medium. Methods for introducing microbial cells into a growth medium are well known in the art. In a preferred embodiment the method includes aseptically introducing the microbial cells into the growth medium.

The strains are introduced into the growth medium in concentrated form, i.e. as a concentrated starter culture or a concentrated starter inoculum. As indicated above, several strains can be co-formulated in which case the concentrated starter culture comprises more than one strain. The concentrated starter culture typically has a content of viable cells (colony forming units, CFU) of at least 1×10⁹ cfu/ml, preferably at least 1×10¹⁰ cfu/ml, more preferably at least 1×10¹¹ cfu/ml, and even more preferably at least 1×10¹² cfu/ml, in particular at least 1×10¹³ cfu/ml. Preferably, the viable cells are cells of lactic acid bacteria.

Concentrated starter cultures can be solid or liquid cultures. Alternatively, the cultures can be a paste or a culture in a transitional glass state, e.g. semi-solid. Solid starter cultures include, but are not limited to, tablets, pellets, capsules, dusts, granules and powders which may be wettable, spray-dried, freeze-dried, air-dried, or lyophilised. Solid starter cultures can be in a frozen state.

Starter cultures can be packaged in any manner that is suitable for providing the cultures to the user, e.g. a bottle, ampoule, container or a bag. The size and weight of the packaging depends among others on the production scale of the dairy plant and can vary from 0.001 kg to 100 kg, preferably 1 kg to 10 kg.

The starter cultures may further comprises at least one additive. A suitable additive includes, but is not limited to, a growth stimulating agent, a stabilising agent, a cryoprotectant, a buffering agent, or a mixture thereof.

Growth stimulating agents include, but are not limited to, yeast extracts, carbohydrates such as sugars, vitamins, hydrolysates, minerals, proteins, peptides, nucleosides, nucleobases, amino acids, nucleotides, and mixtures thereof.

Stabilising agents include, but are not limited to, formic acid, formate, inosinate, inosine, natural and/or chemical antioxidants, nucleosides, nucleobases, amino acids, nucleotides, surfactants such as Tween compounds, hypoxanthine, xanthine, and mixtures thereof.

Cryoprotectant include, but are not limited to, carbohydrates such as sugars, glycols and sugar alcohols, proteins, polyethers, protein hydrolysates, amino acids, and mixtures thereof.

Buffering agents include, but are not limited to, carbonate salts, phosphate salts, citrate salts, magnesium salts, calcium salts, and mixtures thereof. It is to be understood that a derivative of any of the above mentioned compounds should also be considered a suitable additive according to the invention.

The selected bulk starter culture according to the invention has an acidification activity of at least 1.45 ΔpH/2.5 h. In an embodiment the acidification activity is at least 1.50 ΔpH/2.5 h, preferably at least 1.55 ΔpH/2.5 h, more preferably at least 1.60 ΔpH/2.5 h, even more preferably at least 1.65 ΔpH/2.5 h, yet even more preferably at least 1.70 ΔpH/2.5 h, in particular at least 1.75 ΔpH/2.5 h, more in particular at least 1.80 ΔpH/2.5 h, even more particularly at least 1.90 ΔpH/2.5 h and most particularly at least 2.00 ΔpH/2.5 h. “Acidification activity” of the mixed bulk starter culture as used herein means a change (i.e. decrease) in pH of UHT-pasteurised milk having 2% (w/w) fat when inoculated with 3% (v/v) mixed bulk starter culture and incubated for 2.5 hours at 31° C. compared to a non-inoculated control. The acidification activity at 31° C. does not exceed 2.10 ΔpH/2.5 h. Generally, bulk starter cultures are used within 96 hours after preparation. Typically, after 96 hours they rapidly lose acidification activity. Acidification activity is usually measured within 8 hours of completion of the bulk starter culture fermentation. The difference in pH of the UHT-pasteurised milk inoculated with the mixed bulk starter culture of the invention and non-inoculated UHT-pasteurised milk is referred to herein as ΔpH. The mixed bulk starter cultures of the present invention have an acidification activity of at least ΔpH 1.45 after 2.5 hours of cultivation at 31° C. In an embodiment the acidification activity is at least ΔpH 1.50 after 2.5 hours, preferably at least ΔpH 1.55 after 2.5 hours, more preferably at least ΔpH 1.60 after 2.5 hours, even more preferably at least ΔpH 1.65 after 2.5 hours, yet even more preferably at least ΔpH 1.70 after 2.5 hours, in particular at least ΔpH 1.75 after 2.5 hours, more in particular at least ΔpH 1.80 after 2.5 hours, even more particularly at least ΔpH 1.90 after 2.5 hours and most particularly at least ΔpH 2.00 after 2.5 hours of cultivation at 31° C. When the mixed bulk starter culture comprises thermophilic lactic acid bacteria, the acidification activity at 41° C. can also be measured. This activity can be measured by inoculating UHT-pasteurised milk having 2% (w/w) fat with 1% (v/v) mixed bulk starter culture and determining the change (i.e. decrease) in pH of the milk after incubation for a set period of time at 41 ° C. compared to a non-inoculated control.

The growth medium can be any growth medium suitable for the strains to be propagated. It can be a synthetic growth medium (ready-to-use liquid or powdered medium that first has to be reconstituted), whey or milk, to name just a few. In a preferred embodiment the growth medium is milk. “Milk” as used herein includes, but is not limited to, cow milk, human milk, buffalo milk, yak milk, horse milk, zebra milk, camel milk, donkey milk, reindeer milk, goat milk and sheep milk. In a preferred embodiment the milk is cow milk. “Milk” as used herein can be whole milk (about 3.5% (w/w) butterfat), skimmed milk (less than 0.5% (w/w) butterfat), semi-skimmed milk (1.5-1.8% (w/w) butterfat), low fat milk (about 1% (w/w) butterfat), reduced fat milk (about 2% (w/w) butterfat), milk protein concentrate comprising 6-17% (w/w) dry milk solids, or any other kind of milk. It can be pasteurized or non-pasteurized and/or concentrated or non-concentrated milk and/or native or reconstituted milk.

The growth medium can be heat treated before the microbial cells are introduced into the medium. Suitable heat treatments include, but are not limited to, pasteurization methods such as high temperature/short time (HTST) pasteurization (heating medium, e.g. milk, to a temperature of 71° C. to 75° C., for about 15 to 30 seconds); ultra-high temperature (UHT) pasteurization (heating medium, e.g. milk, to a temperature of about 135° C. or higher for around 1-2 seconds); batch or vat pasteurization (heating large batches of medium, e.g. milk, to temperature of typically 63° C. for 30 minutes, followed by quick cooling to about 4° C.); higher heat/shorter time (HHST) pasteurization (pasteurization of medium, e.g. milk, that lies somewhere between HTST and UHT in terms of time and temperature). After the heat treatment the temperature of the growth medium should be adjusted to the temperature suitable for propagation of the cells.

In an embodiment the incubation step is performed under temperature and/or pH control. The pH control can be internally and/or externally, with external pH control being preferred. Internal pH control includes, but is not limited to, the use of buffering agents. External pH control includes, but is not limited to, addition of aqueous ammonia, sodium hydroxide or other food grade caustic. The pH is controlled at a pH of about 5 or higher, preferably between 5.0 and 6.5, preferably above 5.8.

The preparation of the bulk starter culture is done in a starter inoculation tank or vessel. Preferably, the tank or vessel is sanitized, i.e. cleaned and treated with a bactericidal sanitizer solution such as e.g. sodium hypochlorite. In an embodiment the preparation of the bulk starter culture is performed in a large-scale fermentor comprising from 10 to 100,000 litre growth medium, preferably 100 to 10,000 litre growth medium.

In a further aspect the invention is concerned with a mixed bulk starter culture obtainable by a method according to the invention. Details about the mixed bulk starter culture can be found in the above paragraphs. In a preferred embodiment the mixed bulk starter culture is a composition comprising at least two different Lactococcus lactis strains. In an embodiment at least one of the Lactococcus lactis strains is a Lactococcus lactis subsp. cremoris strain.

The mixed bulk starter culture has an acidification activity of at least 1.45 ΔpH/2.5 h at 31° C., preferably at least 1.50 ΔpH/2.5 h, more preferably at least 1.55 ΔpH/2.5 h, even more preferably at least 1.60 ΔpH/2.5 h, yet even more preferably at least 1.65 ΔpH/2.5 h, most preferred at least 1.70 ΔpH/2.5 h, in particular at least 1.75 ΔpH/2.5 h, more in particular at least 1.80 ΔpH/2.5 h, even more particularly at least 1.90 ΔpH/2.5h and most particularly at least 2.00 ΔpH/2.5 h at 31° C. More details about the acidification activity assay can be found in the above paragraphs.

The mixed bulk starter culture composition according to the invention typically comprises a content of viable cells of at least 1×10⁷ cfu/ml, preferably at least 1×10⁸ cfu/ml, more preferably at least 1×10⁹ cfu/ml, even more preferably at least 1×10¹⁰ cfu/ml, yet even more preferably at least 1×10¹¹ cfu/ml, and in particular at least 1×10¹² cfu/ml. Preferably, the viable cells are cells of lactic acid bacteria.

A further aspect of the invention involves a method of making a fermented product, the method comprising the steps of adding a mixed bulk starter culture according to the invention and allowing the mixed bulk starter culture to ferment the substrate to produce a fermented product. In an embodiment the mixed bulk starter culture is prepared by performing a method for preparing a mixed bulk starter culture according to the present invention. When the substrate is cheese milk, the mixed bulk starter culture can be added to the substrate in an amount ranging from 0.1% (w/w) to 5% (w/w), preferably 0.2% (w/w) to 2.0% (w/w) and more preferably 0.3% (w/w) to 0.5% (w/w). The amount added depends on the application, i.e. the type of cheese to be made, the equipment used, the desired speed of the cheese making, etc. The addition of the mixed bulk starter culture to the substrate can be done immediately after its preparation, however, the mixed bulk starter culture can also be stored for e.g. 1 to 72 hours before being added to the substrate. Bulk starter storage is preferably done under cooling to e.g. 4° C. In the addition step the substrate is incubated under conditions favorable to the metabolic activity of the microbial cells, e.g. lactic acid bacteria cells, in the bulk starter culture, so as to obtain the expected and desired fermented product. The invention also relates to a method for manufacturing a food or feed product comprising adding a mixed bulk starter culture according to the invention to a food or feed product starting material and keeping the thus inoculated starting material under conditions where the microbial cells, e.g. lactic acid bacteria, in the mixed bulk starter culture are metabolically active, i.e. have acidification activity. The invention further is concerned with a method for preparing a food or feed product, the method comprising the steps of inoculating a food or feed material with a mixed bulk starter culture according to the invention and incubating the inoculated material under conditions permitting the bulk starter culture to become metabolically active.

The substrate can be a food substrate such as a soya bean substrate, a meat substrate, a substrate for a bakery, wine, beverage, fruit juice or vegetable product or a dairy substrate, e.g. milk-based substrate e.g. milk. Typically, the fermented product is a dairy food product or dairy food-derived product, e.g. cheese, yoghurt, butter, quark, sour cream, matured cream, infant milk, cream dessert, ice cream, inoculated sweet milk, buttermilk, kefir, koumiss, milk beverage, fermented whey-based beverage, fermented milk or drinking yoghurt.

Furthermore, the invention relates to the use of a mixed bulk starter culture according to the present invention for the production of a fermented product. In addition, the invention relates to the use of a mixed bulk starter culture according to the present invention in a process for making a food or feed product.

### EXAMPLES

To illustrate the invention, the following examples are provided. These examples are not intended to limit the scope of the invention.

### Example 1

### Preparation of Concentrated Starter Cultures Used For Preparing Mixed Bulk Starter Cultures

Single strains of Lactococcus lactis ssp. lactis and cremoris were grown and centrifuged at 5000*g for 20 minutes at 5° C. to concentrate the strains. Next, supernatant was decanted from the concentrates and the obtained starter culture concentrates having a content of viable cells ranging from 8*10¹⁰ to 4*10¹¹ cfu/g were mixed with cold cryoprotectant and stored at −60° C. until testing.

The concentrated starter cultures were removed from the freezer, diluted with 2.33 times their weight of cold UHT-pasteurised skimmed milk, mixed until homogeneous, and immediately tested for acid production. Acidification activity was determined by inoculating cold 2%-fat UHT-pasteurised milk with 1.5% (v/v) of the diluted cultures, incubating the inoculated milk for 2.5 hours at 31° C., and then measuring the change in pH of the milk versus non-inoculated milk.

Activity numbers shown in Table 1 represent the drop in the pH of the milk after 2.5 hours (ΔpH/2.5 hours) caused by acid production of the culture. The concentrated starter cultures prepared had a similar acidification activity.

### Example 2

### Preparation of Mixed Bulk Starter Cultures

Mixed bulk starter cultures were prepared by inoculating UHT-pasteurised skimmed milk with about 0.4% (v/v) of the respective concentrated starter cultures. The respective concentrated starter cultures were taken directly from the freezer and introduced separately into the milk. The inoculated milk was incubated for 6.5 hours at 31° C. with ammonia injection to maintain the pH between 5.8 and 6.0, subsequently brought at a temperature of below 10° C. and then tested for acidification activity.

The acidification activity of the mixed bulk starter cultures was tested by directly inoculating 3% (v/v) of the mixed bulk starter cultures into cold 2%-fat UHT-pasteurised milk, incubating the inoculated milk for 2.5 hours at 31° C., and measuring the difference in pH of the milk versus a non-inoculated control.

Activity numbers shown in Table 2 represent the drop in the pH of milk after 2.5 hours (ΔpH/2.5 hours) caused by the acid production of the mixed bulk starter culture.

The results show that mixed bulk starter cultures having an acidification activity of at least 1.45 ΔpH/2.5 hours were prepared. The results further show that mixed bulk starter cultures prepared by introducing at least one Lactococcus lactis subsp. cremoris strain have in general a higher acidification activity compared to mixed bulk starter cultures prepared by introducing only Lactococcus lactis subsp. lactis strains (see Table 2). In order to obtain high-activity bulk starter cultures, it is therefore preferred to include one or more Lactococcus lactis subsp. cremoris strains in the inoculum. Moreover, the results show that mixed bulk starter cultures prepared by introducing at least two different Lactococcus lactis subsp. cremoris strains have in general a higher acidification activity compared to mixed bulk starter cultures prepared by introducing only one Lactococcus lactis subsp. cremoris strain (see Table 2).

TABLE 1
Acidification activity of concentrated strains used to prepare mixed bulk
starter cultures.

| | Acidification activity |
|---|---|
| Strain | of strain (ΔpH/2.5h) |
| Lactococcus lactis subsp. lactis strain 1 | 1.83 |
| Lactococcus lactis subsp. lactis strain 2 | 1.61 |
| Lactococcus lactis subsp. lactis strain 3 | 1.62 |
| Lactococcus lactis subsp. lactis strain 4 | 1.68 |
| Lactococcus lactis subsp. lactis strain 5 | 1.40 |
| Lactococcus lactis subsp. lactis strain 6 | 1.10 |
| Lactococcus lactis subsp. cremoris strain 1 | 1.70 |
| Lactococcus lactis subsp. cremoris strain 2 | 1.72 |
| Lactococcus lactis subsp. cremoris strain 3 | 1.71 |
| Lactococcus lactis subsp. cremoris strain 4 | 1.64 |
| Lactococcus lactis subsp. cremoris strain 5 | 1.40 |
| Lactococcus lactis subsp. cremoris strain 6 | 1.10 |

TABLE 2
Acidification activity of mixed bulk starter cultures.

| | | Acidification activity |
|---|---|---|
| | | of mixed bulk |
| | | starter culture |
| First strain | Second strain | (ΔpH/2.5h) |
| Lactococcus lactis subsp. | Lactococcus lactis subsp. | 1.24 |
| lactis strain 1 | lactis strain 2 | |
| Lactococcus lactis subsp. | Lactococcus lactis subsp. | 1.25 |
| lactis strain 1 | lactis strain 3 | |
| Lactococcus lactis subsp. | Lactococcus lactis subsp. | 1.36 |
| lactis strain 1 | lactis strain 4 | |
| Lactococcus lactis subsp. | Lactococcus lactis subsp. | 1.22 |
| lactis strain 2 | lactis strain 3 | |
| Lactococcus lactis subsp. | Lactococcus lactis subsp. | 1.37 |
| lactis strain 2 | lactis strain 4 | |
| Lactococcus lactis subsp. | Lactococcus lactis subsp. | 1.36 |
| lactis strain 3 | lactis strain 4 | |
| Lactococcus lactis subsp. | Lactococcus lactis subsp. | 1.32 |
| lactis strain 5 | lactis strain 6 | |
| Lactococcus lactis subsp. | Lactococcus lactis subsp. | 1.70 |
| cremoris strain 1 | cremoris strain 2 | |
| Lactococcus lactis subsp. | Lactococcus lactis subsp. | 1.68 |
| cremoris strain 1 | cremoris strain 3 | |
| Lactococcus lactis subsp. | Lactococcus lactis subsp. | 1.68 |
| cremoris strain 1 | cremoris strain 4 | |
| Lactococcus lactis subsp. | Lactococcus lactis subsp. | 1.61 |
| cremoris strain 2 | cremoris strain 3 | |
| Lactococcus lactis subsp. | Lactococcus lactis subsp. | 1.64 |
| cremoris strain 2 | cremoris strain 4 | |
| Lactococcus lactis subsp. | Lactococcus lactis subsp. | 1.63 |
| cremoris strain 3 | cremoris strain 4 | |
| Lactococcus lactis subsp. | Lactococcus lactis subsp. | 1.40 |
| cremoris strain 5 | cremoris strain 6 | |
| Lactococcus lactis subsp. | Lactococcus lactis subsp. | 1.61 |
| lactis strain 1 | cremoris strain 1 | |
| Lactococcus lactis subsp. | Lactococcus lactis subsp. | 1.57 |
| lactis strain 1 | cremoris strain 2 | |
| Lactococcus lactis subsp. | Lactococcus lactis subsp. | 1.54 |
| lactis strain 1 | cremoris strain 3 | |
| Lactococcus lactis subsp. | Lactococcus lactis subsp. | 1.61 |
| lactis strain 1 | cremoris strain 4 | |
| Lactococcus lactis subsp. | Lactococcus lactis subsp. | 1.58 |
| lactis strain 2 | cremoris strain 1 | |
| Lactococcus lactis subsp. | Lactococcus lactis subsp. | 1.57 |
| lactis strain 2 | cremoris strain 2 | |
| Lactococcus lactis subsp. | Lactococcus lactis subsp. | 1.54 |
| lactis strain 2 | cremoris strain 3 | |
| Lactococcus lactis subsp. | Lactococcus lactis subsp. | 1.57 |
| lactis strain 2 | cremoris strain 4 | |
| Lactococcus lactis subsp. | Lactococcus lactis subsp. | 1.57 |
| lactis strain 3 | cremoris strain 1 | |
| Lactococcus lactis subsp. | Lactococcus lactis subsp. | 1.55 |
| lactis strain 3 | cremoris strain 2 | |
| Lactococcus lactis subsp. | Lactococcus lactis subsp. | 1.53 |
| lactis strain 3 | cremoris strain 3 | |
| Lactococcus lactis subsp. | Lactococcus lactis subsp. | 1.51 |
| lactis strain 3 | cremoris strain 4 | |
| Lactococcus lactis subsp. | Lactococcus lactis subsp. | 1.64 |
| lactis strain 4 | cremoris strain 1 | |
| Lactococcus lactis subsp. | Lactococcus lactis subsp. | 1.60 |
| lactis strain 4 | cremoris strain 2 | |
| Lactococcus lactis subsp. | Lactococcus lactis subsp. | 1.56 |
| lactis strain 4 | cremoris strain 3 | |
| Lactococcus lactis subsp. | Lactococcus lactis subsp. | 1.59 |
| lactis strain 4 | cremoris strain 4 | |
| Lactococcus lactis subsp. | Lactococcus lactis subsp. | 1.36 |
| lactis strain 5 | cremoris strain 5 | |
| Lactococcus lactis subsp. | Lactococcus lactis subsp. | 1.40 |
| lactis strain 5 | cremoris strain 6 | |
| Lactococcus lactis subsp. | Lactococcus lactis subsp. | 1.33 |
| lactis strain 6 | cremoris strain 5 | |
| Lactococcus lactis subsp. | Lactococcus lactis subsp. | 1.28 |
| lactis strain 6 | cremoris strain 6 | |

1. A method for preparing a mixed bulk starter culture, the method comprising the steps of:
a) introducing at least two different concentrated Lactococcus lactin strains into a growth medium,
b) incubating the obtained growth medium for less than ten hours to prepare a mixed bulk starter culture, and
c) selecting a mixed bulk starter culture having an acidification activity of at least 1.45 ΔpH/2.5 h at 31° C.

2. Method according to claim 1, characterized in that the growth medium is milk.

3. Method according to claim 1, characterized in that the growth medium is incubated under pH control.

4. Method according to claim 1, characterized in that the at least two different Lactococcus lactis strains are introduced separately or as a co-formulation into the growth medium.

5. Method according to claim 1, characterized in that a further microbial strain is introduced into the growth medium.

6. Method according to claim 1, characterized in that the strains are introduced as a concentrated starter culture comprising 1×10⁹ to 1×10¹² cfu/ml of lactic acid bacteria.

7. Method according to claim 1, characterized in that the starter culture further comprises a growth stimulating agent, a stabilising agent, a cryoprotectant, a buffering agent, or a mixture thereof.

8. A mixed bulk starter culture obtainable by a method according to claim 1.

9. A mixed bulk starter culture according to claim 8, characterized in that at least one of the Lactococcus lactis strains is a Lactococcus lactis subsp. cremoris strain.

10. A mixed bulk starter culture composition according to claim 8 comprising 1×10⁷ to 1×10¹² cfu/ml of lactic acid bacteria.

11. A method of making a fermented product, the method comprising the steps of:
a) adding a mixed bulk starter culture according to claim 8 to a substrate, and
b) allowing the mixed bulk starter culture to ferment the substrate to produce a fermented product.

12. Use of a mixed bulk starter culture according to claim 8 for the production of a fermented product.
